(19) United States
(12) Patent Application Publication
(10) Pub. No.: US 2007/0043054 A1
(43) Pub. Date: Feb. 22, 2007

(54) Enzyme cofactor combination for supplementing pyruvate dehydrogenase and alpha ketogluterate dehydrogenase complexes

(76) Inventor: James R. Howard, Brawley, CA (US)

Correspondence Address: THE WEBB LAW FIRM, P.C., 700 KOPPERS BUILDING, 436 SEVENTH AVENUE, PITTSBURGH, PA, 15219, US

(21) Appl. No.: 10/562,522

(22) PCT Filed: Mar. 15, 2005
(86) PCT No.: PCT/US05/08439

§ 371 Date: Jun. 30, 2006

Related U.S. Application Data
(60) Provisional application No. 60/553,168, filed on Mar. 15, 2004.

(51) Int. Cl. A61K 31/51; A61K 31/455; A61K 31/198

(52) U.S. Cl. 514/250; 514/276; 514/356; 514/561

(57) ABSTRACT

The present invention provides a method and pharmaceutical composition for preventing or treating the development of syndromes related to dysfunctional energy metabolism, such as neuropathy, spontaneous nocturnal muscle cramps associated with neuropathy, seizuring, diabetes mellitus; pediatric hypoglycemia; myopathy; muscle fatigue; muscle spasms; somnolence; reduced mental acuity; exercise intolerance and myocardial insufficiency, which are due to cofactor deficiencies associated with the pyruvate dehydrogenase complex and the alpha-ketogluterate dehydrogenase (a-ketogluterate dehydrogenase) complex in humans or other mammals in need thereof. In particular, a method of preventing or treating at least one syndrome related to defective glucose metabolism in humans or other mammals is provided comprised of administering a combination of enzyme cofactors containing therapeutically effective amounts of thioctic acid, niacinamide, pantothenate, riboflavin and thiamine. Also provided is a pharmaceutical composition comprised of a carrier and the combination of cofactors.

### CROSS REFERENCE TO RELATED APPLICATIONS

The present invention claims priority to U.S. Provisional Application No. 60/553,168, filed Mar. 15, 2004, which is incorporated by reference herein.

### BACKGROUND OF THE INVENTION

1. Field of the Invention

The present invention relates to dysfunctional energy metabolism of glucose in humans and other mammals. More particularly, the present invention relates to a novel combination of metabolic cofactors: thioctic acid, pantothenic acid, niacinamide, riboflavin and thiamin, which is able to restore normal physical and mental activity in humans and other mammals in need thereof.

2. Description of Related Art

All of the cells in the body of animals metabolize; i.e., break down, food into simpler molecules, and, as a result, energy is released that is used to produce adenosine triphosphate (ATP), the main energy currency of cells. The metabolism of food molecules to release ATP energy is a complex process, referred to as cellular respiration. Cellular respiration begins with a metabolic pathway called glycolysis which takes place in the cytosol of cells, does not require oxygen and yields a relatively small amount of ATP. Glycolysis splits apart glucose, a 6-carbon molecule, to yield two 3-C molecules called pyruvic acid.

Following glycolysis, if oxygen is present (aerobic conditions), the next stage of ATP energy production, referred to as oxidative, or aerobic, respiration takes place, which consists of two metabolic pathways, referred to as the Kreb's Cycle (also called the citric acid cycle or the tricarboxylic acid cycle), and Electron Transport.

Aerobic respiration occurs within mitochondria of cells and is far more effective than glycolysis at recovering energy from food molecules, resulting in the majority of ATP energy released from food, up to twenty times more ATP molecules than glycolysis. Thus, only if oxygen is present do the products of glycolysis enter the pathways of aerobic respiration.

If oxygen is not present, then anaerobic respiration takes place in which the pyruvic acid formed during glycolysis is converted to lactic acid, a process known as lactic acid fermentation and which occurs mostly in muscle cells.

During exercise, breathing may not be able to provide the cells of the body with all the oxygen required for aerobic respiration. Thus, when not enough oxygen is delivered to the cells, the cells switch to lactic acid fermentation, which provides muscles with the energy it needs during exercise. However, there are unpleasant side effects to lactic acid fermentation, such as muscle fatigue, pain, cramps and soreness.

Cellular respiration requires the presence of specific protein enzymes to catalyze the individual reactions occurring in glycolysis and aerobic respiration. Most of the enzymes involved in cellular respiration require a nonprotein component, such as cofactors or prosthetic groups, in order to function. Organic cofactors are referred to as coenzymes. Coenzymes are relatively small molecules compared to the protein part of the enzyme, and many of the coenzymes are derived from vitamins, which are essential in the diet of most animals in that they are not synthesized endogenously.

The major substrate metabolized in cellular respiration is glucose. If there is a deficiency of glucose availability, a defect in one or more enzymes needed for the metabolic pathways of cellular respiration, and/or deficits in cofactor or prosthetic groups, this can result in inadequate ATP energy production in the cells of the body.

Enzyme cofactors enhance the efficiency of enzyme catalysis by a magnitude of about 10⁷ to 10¹⁴. Thus, when there is a deficiency of a cofactor, the affected enzyme is unable to function or its efficiency is greatly reduced. Replacing cofactors with other substances, such as cofactor substitutes, still results in impaired enzyme function and diminished ATP energy production. Thus, a deficiency of a cofactor or replacement with a cofactor substitute leads to impaired glucose metabolism and energy deficits, and often results in clinical syndromes.

The study of diseases of energy metabolism commonly referred to as mitochondrial diseases is an emerging specialty in human medicine. Most of these diseases arise from mutation of the mitochondrial genomes and, to a lesser extent, nuclear genes. Such mutations result in specific dysfunctional enzymes in metabolic pathways and in structural changes of mitochondria that disrupt enzyme orientation in metabolic pathways thereby impairing their efficiency. Clinical syndromes presented as a result of dysfunctional energy metabolism depend upon the metabolic pathway affected and the proportion of dysfunctional mitochondria that are affected, as well as upon the specific tissues of the body that are involved. Organs that typically are affected by diseases of energy metabolism are highly differentiated, non-regenerating tissues requiring high levels of oxygen and energy, such as brain and skeletal and heart muscle. Treatment of these diseases is directed to sustaining life by supplementing high levels of metabolic cofactors in an effort to skew metabolism along specific pathways and to provide substrates for the pathways.

As an example, there are two forms of beriberi in humans: neurologic beriberi and cardiologic beriberi. Each form involves different tissues and organs but are related to a deficit in the vitamin thiamine. Other syndromes are related to different enzymes in a metabolic pathway or deficits of their respective cofactors but result in the same syndrome. For example, polioencephalomalacia in ruminant animals typically is related to the interference of pyruvate metabolism in animals that have consumed excess sulfur. Injections or supplementation with thiamine can prevent as well as treat this syndrome. Polioencephalomalacia also may result from intoxication with monensin (a feed additive to promote increased feed efficiency in cattle) for from deficits in trace minerals. It is known that cattle afflicted with polioencephalomalacia as a result of being raised on trace mineral deficient rations respond to trace mineral injections but not to thiamine injections (U.S. Pat. No. 4,335,116).

Syndromes resulting from defective energy metabolism may be very mild and barely perceptible to the victim or may be severe and overwhelming. For physicians and veterinarians, it can be difficult, expensive and impractical to identify defects of specific enzymes that result from deficits of specific cofactors. Thus, there exists a need to provide an inexpensive and convenient way to prevent or treat deficiencies in cofactors, coenzymes and/or prosthetic groups associated with cellular respiration, which deficiencies result in impaired metabolism of glucose and thus impaired ATP energy production.

### SUMMARY OF THE INVENTION

The present invention fulfills this need by providing a method and pharmaceutical composition for preventing or treating the development of syndromes related to dysfunctional energy metabolism due to cofactor deficiencies associated with the pyruvate dehydrogenase complex and the alpha-ketogluterate (α-ketogluterate) dehydrogenase complex, which deficiencies compromise the production of ATP energy in humans or other mammals.

In particular, the present invention provides a method of preventing or treating at least one syndrome related to defective glucose metabolism in humans or other mammals, comprised of administering a combination of enzyme cofactors comprised of therapeutically effective amounts of between about 25 to 85 weight %, preferably about 68 weight % thioctic acid, between about 5 to 25 weight %, preferably about 13.5 weight % niacinamide, between about 5 to 25 weight %, preferably about 13.5 weight % pantothenate, between about 0.5 to 10 weight %, preferably about 2.5 weight % riboflavin and between about 0.5 to 10 weight %, preferably about 2.5 weight % thiamine.

The therapeutically effective amount of the combination of cofactors that is administered to humans or other mammals can range from between about 0.2 to 5 mg/kg body weight daily.

The present invention also provides a pharmaceutical composition for the treatment of at least one syndrome related to defective glucose metabolism in humans or other mammals in need thereof, comprising an excipient or carrier and therapeutically effective amounts of thioctic acid, niacinamide, calcium pantothenate, riboflavin and thiamine. The pharmaceutical composition can be inserted into capsules, pressed into tablets or produced as a powder or food to be incorporated into the diet.

The combination of cofactors of the present invention can be administered via various routes, including, without limitation, oral or parenteral administration. When orally administered, the combination of cofactors can be in hard or soft shell gelatin capsules, compressed into tablets, sachets, lozenges, elixirs, suspensions, syrups, in the form of a powder or granule, a solution or suspension in an aqueous liquid or non-aqueous liquid, or in an oil-in-water or water-in-oil emulsion. When the combination of cofactors is in the form of a powder or granule, it can be added to the food of humans or other mammals. For example, a powdered mixture of the combination of cofactors of the present invention can be prepared in a loose form to be top-dressed on pet foods or mixed into diets or such dietary constituents as pet treats or energy snack foods for humans.

The combination of cofactors also can include, without limitation, preservatives, stabilizers, anti-caking agents, coloring agents, flavoring agents or combinations thereof.

Syndromes related to defective glucose metabolism that can be treated according to the method of the present invention can include, without limitation, neuropathy; spontaneous muscle cramps, such as nocturnal muscle cramps associated with neuropathy; spinal motor neuropathies; seizuring, such as spontaneous epileptiform seizuring; diabetes mellitus; pediatric hypoglycemia; myopathy; muscle weakness; muscle soreness associated with exercise; muscle spasms; somnolence; memory deficit; reduced mental acuity; exercise intolerance or myocardial insufficiency.

### DETAILED DESCRIPTION OF THE INVENTION

The present invention provides a method and pharmaceutical composition for preventing or treating at least one syndrome related to defective glucose metabolism in humans or other mammals, comprised of the administration of a specific combination of physiological levels of cofactors, such as vitamins and prosthetic groups, which join with enzymes of the pyruvate dehydrogenase complex and alpha ketogluterate (α-ketogluterate) dehydrogenase complex to facilitate ATP energy production in syndromes related to dysfunctional energy metabolism involving these enzyme complexes.

The syndromes prevented or treated according to the method and pharmaceutical composition of the present invention can arise singly or can complicate diseases associated with advancing age or nuclear and/or mitochondrial genome mutation, and can include, without limitation, neuropathy; spontaneous muscle cramps, such as nocturnal muscle cramps associated with neuropathy; spinal motor neuropathies; seizuring, such as spontaneous epileptiform seizuring; diabetes mellitus; pediatric hypoglycemia; myopathy; muscle weakness; muscle soreness associated with exercise; muscle spasms; somnolence; memory deficit; reduced mental acuity; exercise intolerance or myocardial insufficiency. Furthermore, associated heritable enzymatic defects can be compensated for by the administration of this combination of enzyme cofactors.

The method and pharmaceutical composition of the present invention is not intended to be limited to the above-described syndromes and can extend to other syndromes related to dysfunctional energy metabolism that respond to daily consumption of the cofactor combination described herein.

The present invention is intended for use in all mammalian species, and preferably, in addition to humans, can include, without limitation, dogs, cats, cattle and the like.

As used herein, the term “cofactors” refers to vitamins, trace minerals and prosthetic groups involved in the pyruvate dehydrogenase complex and the α-ketogluterate dehydrogenase complex.

As used herein, the term “vitamins” refers to niacinamide, pantothenate, riboflavin, thiamine and their chemically related compounds that are essential to and intimately involved with enzymes in the pyruvate dehydrogenase complex and/or the α-ketogluterate dehydrogenase complex.

As used herein, the term “prosthetic group” refers to thioctic acid, which facilitates the function and contributes to the structure of the pyruvate dehydrogenase complex and the α-ketoglutarate dehydrogenase complex.

As used herein, the term “syndrome” refers to a group of signs or symptoms that typify a particular disease, disorder or condition that varies from normal.

In one embodiment of the present invention, a method is provided to prevent or treat at least one syndrome related to defective glucose metabolism in humans or other mammals, comprised of administering therapeutically effective amounts of a combination of enzyme cofactors containing between about 25 to 85 weight %, preferably about 68 weight % thioctic acid, between about 5 to 25 weight %, preferably about 13.5 weight % niacinamide, between about 5 to 25 weight %, preferably about 13.5 weight % calcium pantothenate, between about 0.5 to 10 weight %, preferably about 2.5 weight % riboflavin and between about 0.5 to 10 weight %, preferably about 2.5 weight % thiamine.

The therapeutically effective amount of the preparation that is administered to humans or other mammals can range from between about 0.2 to 5 mg/kg body weight daily.

In another embodiment of the present invention, a pharmaceutical composition is provided comprised of a carrier and therapeutically effective amounts of a combination of enzyme cofactors containing between about 25 to 85 weight %, preferably about 68 weight % thioctic acid, between about 5 to 25 weight %, preferably about 13.5 weight % niacinamide, between about 5 to 25 weight %, preferably about 13.5 weight % calcium pantothenate, between about 0.5 to 10 weight %, preferably about 2.5 weight % riboflavin and between about 0.5 to 10 weight %, preferably about 2.5 weight % thiamine.

The carrier of the pharmaceutical composition can be any pharmaceutically acceptable carrier or diluent.

The combination of cofactors of the present invention can be administered via various routes, including, without limitation, oral or parenteral administration. When orally administered, the combination of cofactors can be in hard or soft shell gelatin capsules, compressed into tablets, sachets, lozenges, elixirs, suspensions, syrups, in the form of a powder or granule, a solution or suspension in an aqueous liquid or non-aqueous liquid, or in an oil-in-water or water-in-oil emulsion. When the combination of cofactors is in the form of a powder or granule, it can be added to the food of humans or other mammals. For example, a powdered mixture of the combination of cofactors of the present invention can be prepared in a loose form to be top-dressed on pet foods or mixed into diets or such dietary constituents as pet treats or energy snack foods for humans.

The combination of cofactors also can include, without limitation, preservatives, stabilizers, coloring agents, flavoring agents or combinations thereof.

All preparations of the combination of cofactors of the invention are easy, safe and convenient to use. The liquid for oral consumption can be taken directly into the mouth and swallowed or measured into it with a spoon, dropper, syringe, or like device. Similarly, the liquid preparation can be measured into food or drink for consumption. The usual, standardized techniques for parenteral injection of a drug with hypodermic needle and syringe can be employed for administering the injectable form of the invention subcutaneously, intramuscularly, intravenously, or as an additive to compatible liquid medicaments designed for intravenous injection.

The combination of cofactors of the present invention is easy to prepare. Liquids for oral use are prepared at room temperature by dissolving prescribed quantities of crystalline forms of the cofactors in water, adding preservative and coloring and/or flavoring, filter sterilizing, and bottling. Liquid for injection is prepared at room temperature by dissolving prescribed amounts of each cofactor in water. If the material is to be dispensed in a multi-dose vial, preservative is added before the pH is adjusted with NaOH to neutrality and the solution is filter sterilized and bottled. Dry forms of the invention are prepared by mixing prescribed amounts of the desiccated cofactors. If the invention is to be encapsulated, an anticaking agent to facilitate production may be added prior to encapsulation. If the dry preparation is to be dissolved for intravenous injection the desecrated powder or crystalline mixture can be measured into glass vials, sealed and sterilized.

The combination of cofactors of the present invention include four water-soluble vitamins: niacinamide, pantothenate, riboflavin and thiamine; and one prosthetic group, thioctic acid.

Thioctic acid has the following chemical structure:

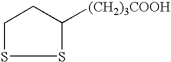

Thioctic acid (also known as α-lipoic acid, 1,2-dithiolane-3-pentanoic acid, 1,2-ditholane-3-valeric acid, 6,8-thiotic acid; 5-[3-C1,2-dithiolanyl)]-pentanoic acid; delta-[3-(1,2-dithiacyclopentyl)] pentanoic acid, acetate replacing factor and pyruvate oxidation factor) is a disulfide compound that is a cofactor in vital energy-producing reactions in the body. It is also a potent biological antioxidant. Thioctic acid once was thought to be a vitamin for animals and humans, however, it is now known to be made endogenously in humans, and so it is not an essential nutrient. There are, however, certain situations, for example, in diabetic polyneuropathy, where thioctic acid may have conditional essentiality. Most of the metabolic reactions in which α-lipoic acid participates occur in mitochondria. These include the oxidation of pyruvic acid (as pyruvate) by the pyruvate dehydrogenase enzyme complex and the oxidation of alpha-ketoglutarate by the alpha-ketoglutarate dehydrogenase enzyme complex. It is also a cofactor for the oxidation of branched-chain amino acids (leucine, isoleucine and valine) via the branched-chain α-keto acid dehydrogenase enzyme complex.

Niacinamide has the following chemical structure:

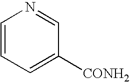

Niacinamide is the physiologically active form of niacin. Niacinamide is a crystalline powder soluble in water and ethanol, and the dry form of niacin is stable up to about 60° C. In aqueous solutions, it is stable for a short period when autoclaved. Niacinamide is the form in which niacin is found in niacinamide adenine dinucleotide (NAD), and in niacinamide adenine dinucleotide phosphate (NADP).

The major function of niacinamide in NAD and NADP is hydrogen transport in intermediary metabolism. Most of these enzyme systems function by alternating between the oxidized and reduced state of the coenzymes NAD-NADH and NADP-NADPH. Both NAD and NADP are involved in the synthesis of high energy phosphate bonds which furnish energy for certain steps in glycolysis, in pyruvate metabolism and in amino acid and protein metabolism.

Pantothenate (pantothenic acid) has the following chemical structure:

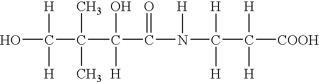

Pantothenic acid is essentially dihydroxydimethylbutyric acid bonded to β-alanine. The free acid is a yellow, viscous oil, whereas the salt is a white crystalline powder readily soluble in water, and is almost insoluble in organic solvents. It is stable to oxidizing and reducing agents and to autoclaving, but is labile to dry heat, hot alkali, or hot acid.

Pantothenic acid is part of acetyl Co-A, and thus is required by all animal species and by many microorganisms. The acetyl Co-A system is involved in the acetylation of aromatic amines and choline; condensation reactions for synthesis of acetate, fatty acids, and citrate; the oxidation of pyruvate and acetaldehyde; and is essential for the development of the central nervous system. Acetyl Co-A also is involved in acylation of acetate, succinate, benzoate, propionate and butyrate. Pantothenic acid is involved in adrenal function and for the production of cholesterol.

Thiamine has the following chemical structure:

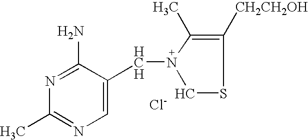

Thiamine is a water-soluble, colorless, crystalline compound. It is comparatively stable to dry heat but is rapidly broken down in neutral or alkaline solutions and is split by sulphites into constituent pyrimidine and thiazole moieties. It has a characteristic yeast-like odor. The pyrimide ring is relatively stable, but the thiazole ring is easily opened by hydrolysis. Several derivatives are stable to heat and appear to be more completely soluble in weak alkaline solutions than thiamine itself and still show biological activity in animals. These derivatives include thiamine propyl disulphide, benzoylthiamine disulphide, dibenzoylthiamine, and benzoylthiamine monophosphate. Thiamine functions in all cells as the coenzyme cocarboxylase, thiamine pyrophosphate, which participates in the oxidative decarboxylation of pyruvic acid to acetate for entry into the Kreb's Cycle. Thiamine is essential for good appetite, normal digestion, growth and fertility. It is needed for normal functioning of the nervous tissue and its requirement in the diet is determined by the caloric density of the diet.

Riboflavin has the following chemical structure:

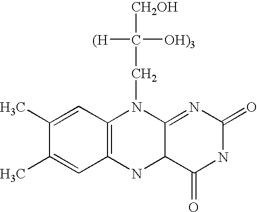

Riboflavin is a yellow-brown crystalline pigment. The vitamin is very slightly soluble in water but soluble in alkali. It is insoluble in most organic solvents. Riboflavin is stable to oxidizing agents in strong mineral acids and in neutral aqueous solution. It also is stable to dry heat but is irreversibly decomposed on irradiation with ultraviolet rays or visible light, breaking down to lumiflavin. Riboflavin functions in the tissues in the form of flavin adenine dinucleotide (FAD) or as flavin mononucleotide (FMN). The flavo-proteins function as enzymes in cellular respiration and are involved in hydrogen transport to catalyze the oxidation of reduced pyridine nucleotides AH and NADPH). Thus, they function as coenzymes for many oxidases and reductases, such as cytochrome e reductase, D- and L-amino acid oxidases, xanthine and aldehyde oxidase, succinic dehydrogenase, glucose oxidase and fumaric dehydrogenase. Riboflavin also is involved with pyridoxine in the conversion of tryptophan to nicotinic acid and is important in the cellular respiration of poorly vascularized tissues, such as the cornea of the eye. Riboflavin is involved in the retinal pigment during light adaptation and the lack of this vitamin causes impaired vision and photophobia in animals.

All of the cofactors of the present invention are involved in the proper functioning of enzymes that are required in the pyruvate dehydrogenase complex and the α-ketogluterate dehydrogenase complex. In particular, the pyruvate dehydrogenase complex is involved in the synthesis of acetyl Co-A from pyruvic acid, which occurs in the transition reaction. The transition reaction precedes the Kreb's Cycle and occurs if oxygen is present. In the transition reaction, pyruvic acid is broken down to remove one carbon and two oxygens, forming carbon dioxide. When the carbon dioxide is split off from pyruvic acid, energy is released, and NAD⁺ is converted into the higher energy form NADH. Coenzyme A attaches to the remaining 2-C (acetyl) unit, forming acetyl Co-A. This process is a prelude to the Kreb's Cycle.

The α-ketogluterate dehydrogenase complex is part of the Kreb's Cycle. In the Kreb's Cycle, the acetyl Co-A (2-C) is attached to a 4-C chemical (oxaloacetic acid). The Co-A is released and returns to await another pyruvic acid. The 2-C and 4-C bond to make citric acid, a 6-C molecule. The metabolic steps that occur after citric acid is formed essentially splits off more carbon dioxide from the molecules and releases energy in the form of ATP, GTP, NADH and FADH₂. Between isocitric acid and α-ketoglutaric acid, carbon dioxide is given off and NAD⁺ is converted into NADH. Between α-ketoglutaric acid and succinic acid, the release of carbon dioxide and reduction of NAD⁺ into NADH again occurs, resulting in a 4-C chemical, succinic acid. GTP (guanine triphosphate), which transfers its energy to ATP, also is formed.

The remaining energy carrier-generating steps involve the shifting of atomic arrangements within the 4-C molecules. Between succinic acid and fumaric acid, the molecular shifting does not release enough energy to make ATP or NADH outright, but instead energy is captured by a new energy carrier, flavin adenine dinucleotide (FAD). FAD is reduced by the addition of two H's to become FADH2. FADH2 is not as rich an energy carrier as NADH, yielding less ATP than NADH.

The last step, between malic acid and oxaloacetic acid, reforms oxaloacetic acid to complete the cycle. Energy is given off and trapped by the reduction of NAD⁺ to NADH.

The method and pharmaceutical composition of the present invention for preventing and treating at least one syndrome related to defective glucose production in humans and other mammals will be described in more detail in the following non-limiting examples.

### EXAMPLE 1

### Exercise Intolerance in a Geriatric Patient Due to Defective Energy Metabolism

Over a course of several months, a geriatric patient had developed exercise intolerance. This disability was characterized by a reduced ability to maintain a brisk walking speed at all times but was especially noticeable when going up gradual grades. Thus, frequent rest stops were required when walking up hills. It was believed that the exercise intolerance was due to deficient energy availability. Additionally, the patient suffered from low-grade muscle pain that developed and persisted throughout exercising in the major muscles of both legs.

Thioctic acid, in a dose of 100 mg once daily, was administered orally to the patient. The patient experienced an improvement in exercise tolerance and a decrease in muscle pain. However, recovery of normal function of the major muscles of both legs was not attained until niacinamide, pantothenate, thiamine and riboflavin were added two months after commencement of the thioctic acid administration regimen.

Muscle burning and pain during physical exertion are characteristic of lactic acid accumulation in the muscle. In instances of extreme exertion, anaerobic conditions typically develop and energy demands become dependent upon relatively energy-inefficient glycolysis. This results in the over-production of pyruvic acid which then is reduced to lactic acid. The exercise-intolerant geriatric patient did not have adequate ATP energy needed for extreme muscle exertion that is associated with anaerobic respiration, i.e., anaerobiosis. However, the patient experienced muscle weakness and reduced endurance with muscle pain and burning during the mild exertion of walking, typically an aerobic respiratory activity; i.e., aerobiosis, suggesting that an anaerobic-like condition was created as the muscles, during walking, depleted typical energy sources and became dependent upon glycolysis. This syndrome of muscle weakness, pain and burning upon mild exertion of the muscles was ameliorated by administering to the patient a combination of thioctic acid, niacinamide, pantothenate, riboflavin and thiamin, all of which are cofactors to enzymes of the pyruvate dehydrogenase complex. The amelioration of the syndrome suggests that there was a cofactor deficit or defect of the pyruyate dehydrogenase complex that restricted the conversion of pyruvic acid to acetyl coA and interrupted the metabolic progression to oxidative respiration, i.e., phosphorylation, in the mitochondria, and thus resulted in limited ATP energy production. The resultant energy limitations necessitated increased glycolyis for ATP production that resulted in a build up of pyruvic and lactic acids. The muscle weakness and reduced exercise endurance experienced by this patient, therefore, was a result of an energy deficit due to the inability of cells to process pyruvate through the Kreb's Cycle.

The response of the geriatric patient to the cofactor combination preparation was surprising, in that more than a single cofactor was required in order to ameliorate the muscle weakness syndrome. This demonstrates that the syndrome probably was related to defects in more than a single enzyme in the pyruvate dehydrogenase complex. The partial response of the syndrome to thioctic acid administration suggests a deficit of this prosthetic group, however, the cause of this deficit is unknown. The enzyme deficit may have been age-related due to reduced synthesis or increased excretion of thioctic acid or to an alteration of enzyme structure to a form that required more thioctic acid. Because the addition of the four vitamins were required to fully ameliorate the syndrome points to a deficiency of one or more vitamins that may be related to the patients' age.

Furthermore, the possibility exists that the impairment in energy metabolism observed in this patient may have been related to a defective α-ketogluterate dehydrogenase complex, resulting in reduced conversion of ketoglutarate to succinyl-CoA in the Kreb's Cycle. Syndromes that are related to the pyruvate dehydrogenase complex and to the α-ketogluterate dehydrogenase complex are clinically similar in that both result in muscle weakness and lactic acid accumulation. Structurally and functionally, both complexes have a need for large amounts of thioctic acid and both complexes have similar enzyme and vitamin requirements.

### EXAMPLE 2

### Nocturnal Muscle Cramping in Two Geriatric Patients Due to Defective Energy Metabolism

Over a course of a year, two geriatric patients developed painful nocturnal muscle cramping asymmetrically distributed in their leg muscles; i.e., the cramping did not cause contraction of the entire leg muscle mass. This probably was because, while small groups of fibers within the leg muscle were contracting painfully, the bulk of the muscle fibers remained relaxed. However, any change in muscle tension during the spasm increased the pain. The apparent pattern of muscle fiber involvement was similar to the histopathologic distribution of muscle fiber atrophy typically observed after individual nerve fibers are destroyed, suggesting that the spasmed muscle fibers were innervated by neurons irritated or injured as a consequence of metabolic disturbances. The muscle cramping also occasionally developed during waking hours when the patients were walking or when at rest. Additionally, one of the patients experienced cramping of the hands while working, which caused difficulty in grasping and releasing objects. The frequency and severity of the cramping was increased when more than usual amounts of sugar were consumed the previous day.

Both patients were given 100 mg thioctic acid daily and the cramping episodes were reduced approximately 50% in both frequency and severity in both patients. When thioctic acid was combined with niacinamide, pantothenate, riboflavin and thiamine, all cramping symptoms were reduced to minor isolated episodes of relatively painless spasm of the muscles occurring at five to seven day intervals. When the administration of the combination of cofactors was stopped, full-blown symptoms of cramping resumed after three days, but again ceased when the administration of the combination of cofactors was resumed.

The syndrome's random fiber involvement and association with increased sugar consumption, as well as its amelioration by the administration of the combination of cofactors indicated dysfunctional glucose metabolism of peripheral nerve cells. The administration of the combination of cofactors was able to correct or compensate for this metabolic defect.

### EXAMPLE 3

### Seizures in an Australian Shepard Dog Due to Defective Energy Metabolism

Over a period of one year, a 12-year old Australian Shepherd bitch had been experiencing seizures with increasing frequency. She previously had been treated with a combination of L-carnitine, acetyl-L-carnitine, pantothenate and niacinamide, as disclosed in U.S. Pat. No. 5,977,004, along with primidone or potassium bromide. This treatment was stopped and she then was given a 110 mg preparation of a combination of cofactors consisting of thioctic acid, niacinamide, pantothenate, riboflavin and thiamine daily. All seizuring ceased and her level of physical activity and mental acuity increased to that which it had been three years previously. This demonstrated that some seizuring syndromes might benefit from supplementing the diet with this combination of cofactors involved in the pyruvate dehydrogenase complex and the O-ketogluterate dehydrogenase complex. Moreover, this cofactor combination may have usefulness for the management of some brain dysfunction syndromes.

The above three examples demonstrate that some syndromes characterized by skeletal muscle weakness and/or peripheral nerve or brain dysfunction are amenable to treatment with cofactors to enzymes of the pyruvate dehydrogenase complex and the C-ketogluterate dehydrogenase complex. In particular, the skeletal muscle weakness syndrome of the patient described in Example 1 showed signs of compensatory glycolysis accompanied with lactic acid accumulation. Similar syndromes that involve skeletal muscle, peripheral nerves and brain, which also are related to dysfunctional energy metabolism, have been reported in U.S. Pat. Nos. 5,889,055 and 5,973,004. The syndromes reported therein, however, have a different underlying enzyme or cofactor defect, as they respond to treatment with a combination of L-carnitine, acetyl-L-carnitine, niacinamide, and pantothenic acid and do not manifest lactic acid accumulation. Furthermore, the patient syndromes reported in the three examples provided herein were treated unsuccessfully with the combination of L-carnitine, acetyl-L-carnitine, niacinamide, and pantothenic acid prior to administration of the cofactor combination of the present invention, namely, thioctic acid, niacinamide, pantothenate, riboflavin and thiamine. Additionally, syndromes in the beriberi group as well as pellagra also should be amenable to prevention and treatment with the method and pharmaceutical composition of the present invention.

While the invention has been described in connection with what is presently considered to be the most practical embodiments, it is to be understood that the invention is not to be limited to the disclosed embodiments, but on the contrary, is intended to cover various modifications and equivalent arrangements included within the spirit and scope of the following claims.

The invention claimed is:

1. A method of preventing or treating at least one syndrome related to defective glucose metabolism in humans or other mammals in need thereof, comprising administering to the mammal or human a therapeutically effective amount of a combination of cofactors comprising thioctic acid, niacinamide, pantothenate, riboflavin and thiamine.

2. The method of claim 1, wherein the combination of cofactors comprises about 25 to 85 weight % thioctic acid, between about 5 to 25 weight % niacinamide, between about 5 to 25 weight % pantothenate, between about 0.5 to 10 weight % riboflavin and between about 0.5 to 10 weight % thiamine.

3. The method of claim 1, wherein the combination of cofactors comprises about 68 weight % of thioctic acid, about 13.5 weight % of niacinamide, about 13.5 weight % of calcium pantothenate, about 2.5 weight % of riboflavin and about 2.5 weight % of thiamine.

4. The method of claim 1, wherein the therapeutically effective dosage amount of the combination of cofactors administered to human or other mammals ranges from between about 0.2 to 5 mg/kg body weight daily.

5. The method of claim 1, wherein the at least one syndrome to be treated is selected from the group consisting of neuropathy; spontaneous muscle cramps, such as nocturnal muscle cramps associated with neuropathy; spinal motor neuropathies; seizuring, such as spontaneous epileptiform seizuring; diabetes mellitus; pediatric hypoglycemia; myopathy; muscle weakness; muscle soreness associated with exercise; muscle spasms; somnolence; memory deficit; reduced mental acuity; exercise intolerance and myocardial insufficiency.

6. The method of claim 1, wherein the mammals are selected from the group consisting of domesticated dogs, cats and cattle.

7. The method of claim 1, wherein the defective glucose metabolism includes defective pyruvate dehydrogenase complex activity and α-ketogluterate dehydrogenase complex activity.

8. The method of claim 1, wherein the route of administration of the preparation to the human or other mammal is via oral or parenteral administration.

9. The method of claim 8, wherein the oral administration is selected from the group consisting of hard or soft shell gelatin capsules, tablets, sachets, lozenges, elixirs, suspensions, syrups, powders, granules, solutions or suspensions in aqueous liquid or non-aqueous liquid and oil-in-water or water-in-oil emulsion.

10. The method of claim 9, wherein the powder or granules is added to food.

11. The method of claim 9, wherein the powder or granules are mixed with dietary constituents, such as energy snacks for humans or pet treats.

12. A method as recited in claim 8, wherein the parenteral administration is selected from the group consisting of intravenous, subcutaneous and intramuscular.

13. The method of claim 1, wherein the combination of cofactors includes additives selected from the group consisting of preservatives, stabilizing agents, anti-caking agents, coloring agents, flavoring agents and combinations thereof.

14. A pharmacological composition for preventing or treating at least one syndrome related to defective glucose metabolism in humans or mammals in need thereof, comprising a carrier and the combination of cofactors as recited in claim 1.
